(19) United States
(12) Patent Application Publication
(10) Pub. No.: US 2022/0000787 A1
(43) Pub. Date: Jan. 6, 2022

(54) DELAYED RELEASE PHARMACEUTICAL COMPOSITION OF PREDNISONE AND PREPARATION THEREOF

(71) Applicant: PIRAMAL PHARMA LIMITED, Mumbai (IN)

(72) Inventors: Umesh V. BARABDE, Ahmedabad (IN); Narendra PATEL, Ahmedabad (IN); Dinesh M. MORKHADE, Ahmedabad (IN); Bhushan Santoshrao BHOYAR, Ahmedabad (IN)

(73) Assignee: PIRAMAL PHARMA LIMITED, Mumbai (IN)

(21) Appl. No.: 17/363,238

(22) Filed: Jun. 30, 2021

(30) Foreign Application Priority Data
Jul. 1, 2020 (IN) 202021028081

(51) Int. Cl. A61K 9/20; A61K 31/573; A61K 9/28

(52) U.S. Cl. CPC A61K 9/2095; A61K 31/573; A61K 9/2833; A61K 9/2086; A61K 9/2013; A61K 9/2027; A61K 9/2009

(57) ABSTRACT

The present invention relates to a delayed-release pharmaceutical composition comprising an active ingredient prednisone and one or more pharmaceutical excipient(s). The invention further relates to a process for preparation of said pharmaceutical composition for oral administration, particularly a tablet, comprising prednisone with one or more pharmaceutically acceptable excipient(s), wherein the tablet is formulated using a coating technique which has a significant impact on drug release.

### FIELD OF THE INVENTION

The present invention relates to a delayed-release pharmaceutical composition comprising an active ingredient prednisone and one or more pharmaceutical excipient(s). The invention further relates to a process for preparation of said pharmaceutical composition for oral administration, particularly a tablet, comprising prednisone with one or more pharmaceutically acceptable excipient(s), wherein the tablet is formulated using a coating technique which has a significant impact on drug release.

### BACKGROUND OF THE INVENTION

Prednisone belongs to a class of drugs known as corticosteroids. Corticosteroids are adrenocortical steroids, both naturally occurring and synthetic. The molecular formula for prednisone is C₂₁H₂₆O₅. The chemical name for prednisone is 17,21-dihydroxypregna-1,4-diene-3,11,20-trione, and the structural formula is:

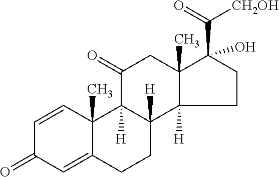

Prednisone is a white to practically white, odorless, crystalline powder and has a molecular weight of 358.43. Prednisone is very slightly soluble in water; slightly soluble in alcohol, chloroform, dioxane, and methanol. Prednisone is used as an anti-inflammatory or an immunosuppressant medication. It treats many different conditions such as allergic disorders, skin conditions, ulcerative colitis, arthritis, lupus, psoriasis, or breathing disorders.

RAYOS® a delayed-release prednisone tablet marketed in U.S. is intended for oral administration. It consists of a prednisone-containing core tablet in an inactive shell, which delays the onset of in vitro drug dissolution by approximately 4 hours. Each tablet contains 1 mg, 2 mg, or 5 mg of prednisone, with the following inactive ingredients: dibasic calcium phosphate dihydrate, colloidal silicon dioxide, croscarmellose sodium, glycerol dibehenate, lactose monohydrate, magnesium stearate, povidone, yellow ferric oxide, and red ferric oxide.

U.S. Pat. No. 8,168,218 provides a press-coated tablet comprising a core containing an drug substance, and a coating, the core being disposed within the coating such that the coating has a first thickness about an axis A-B and a thickness about an orthogonal axis X-Y, such that the coating about the axis X-Y is thicker than the coating about the axis A-B, and is adapted to provide a lag time of between about 2 to 6 hours during which substantially no drug substance is released.

PCT Publication No. WO2016114726 relates to a time-controlled delayed release tablet comprising prednisone whose release active ingredient from core after a lag time wherein said, time-controlled delayed release tablets are press-coated tablets comprising a core and a coating covering said core.

U.S. Publication No. 20130243861 claims a press-coated tablet comprising a core comprising prednisone, and a coating around the core, said core being disposed within said coating such that the coating thickness about an axis (A-B) is equal to or thicker than the coating thickness about an axis (X-Y) orthogonal to (A-B), wherein the axis (A-B) is the axis of the direction of movement of the punch used in the press-coating of the tablet.

Drug Design, Development and Therapy; 2016:10, 1047-1058, Marco Krasselt et al, “Efficacy and safety of modified-release prednisone in patients with rheumatoid arthritis” reports that the existing data on MR prednisone suggest a superior efficacy in reducing RA-related morning stiffness while bearing a safety profile comparable to conventional prednisone formulations.

Rheumatol Ther (2017) 4:363-374, Ara H. Dikranian et al., “Switching From Immediate-Release to Delayed-Release Prednisone in Moderate to Severe Rheumatoid Arthritis: A Practice-Based Clinical Study” analyzes the incremental benefit of better timed and lower dose glucocorticoid therapy.

Rheumatoid arthritis (RA) is a chronic inflammatory joint disease that can cause cartilage and bone destruction if left untreated. Upon confirming a diagnosis of RA, a variety of treatment approaches are typically considered, including non-steroidal anti-inflammatory drugs (NSAIDs), synthetic disease-modifying anti-rheumatic drugs (DMARDs), biologic DMARDs, and glucocorticoids.

Patients with RA—a disease that can painfully swell joints—often wake to crippling stiffness caused by an overnight increase in inflammation. Since many RA medications activate within one to two hours, patients must take their prescriptions in the early morning to minimize pain. RA is commonly treated with low-dose immediate-release prednisone (IR-prednisone) in the morning upon awakening. This timing may not optimize control of inflammation and disease symptoms because inflammatory cytokines peak before awakening, typically around 2-4 a.m. Administering a delayed-release prednisone (DR-prednisone) tablet at bedtime enables timed delivery of prednisone before and during this circadian peak of inflammatory cytokine release and thereby potentially influences disease symptoms.

Hence it is important that the drugs that work well for the disease need to be present in the body in the early morning hours. The concept of time-controlled or delayed release formulations are known in the art that are able to deliver drug substances with a defined release rate after a lag time during which no drug substance is released. The coating acts as a barrier to the ingress of aqueous media into an active-agent-containing core and thereby creating a lag time during which no drug substance is released. The coating act as a medium through which drug is released in a delayed or modified manner. These formulations are completely independent of pH. In other words, release the active ingredient after a lag time regardless of the physiological pH environment in the gastrointestinal tract.

There have been several time-release, delayed-release or modified-release formulations techniques reported in the art. Nowadays, to develop modified-released products, the Tablet in Tablet technology is the best alternative for bilayer tablet formulation for the incompatible drug. It involves the compression of granular materials around a preformed tablet core using specially designed tableting equipment. This methodology appears to be highly cumbersome and costly. Also, there are some new dry-coated devices for the release of drug after a programmable period of time. It is intended to be used mainly in the therapy of those diseases which depend on circadian rhythms. However, there is a need to have a delayed-release formulation technique that is less complex, economical and viable.

In consideration of the need as indicated above, the inventors of present invention has carried out extensive research to evaluate the right process, and right excipients to ultimately give a robust, high productivity and regulatory compliant product of good quality.

### SUMMARY OF THE INVENTION

In one aspect, the present invention relates to a delayed-release pharmaceutical composition comprising prednisone and one or more pharmaceutical excipient(s).

In yet another aspect, there is provided a delayed-release pharmaceutical composition comprising prednisone, one or more diluent(s), one or more binder(s), one or more disintegrant(s), one or more lubricant(s), one or more glidant(s) and one or more coating material(s).

In another aspect, there is provided a process for the preparation of a delayed-release pharmaceutical composition comprising prednisone and one or more pharmaceutical excipient(s).

In another aspect, there is provided a process for the preparation of a delayed-release pharmaceutical composition comprising prednisone with one or more pharmaceutically acceptable excipient(s), wherein the tablet is formulated using a coating technique which has a significant impact on drug release.

In another further aspect, there is provided a method for treating or preventing various conditions, diseases, disorders, comprising administering to a subject in need thereof any one of the compositions of the present invention in an amount effective to treat or prevent a condition, a disease or a disorder.

In another further aspect, there is provided use of a delayed-release pharmaceutical composition comprising prednisone and one or more pharmaceutical excipient(s), for the manufacture of a medicament for treating or preventing various conditions, diseases or disorders.

In a still further aspect, the present invention relates to a pharmaceutical kit comprising: (a) prednisone and one or more pharmaceutical excipient(s); and (b) optionally a package insert comprising instructions for using the said pharmaceutical composition.

These and other aspects and advantages of the present invention will be apparent to those skilled in the art from the following description.

### BRIEF DESCRIPTION OF DRAWINGS OF THE INVENTION

FIG. 1 represents Comparative drug release profile of Prednisone DR Tablets Test formulations with Reference product provided in Table 3.

FIG. 2 represents Comparative drug release profile of Prednisone DR Tablets Test formulations with Reference product provided in Table 5.

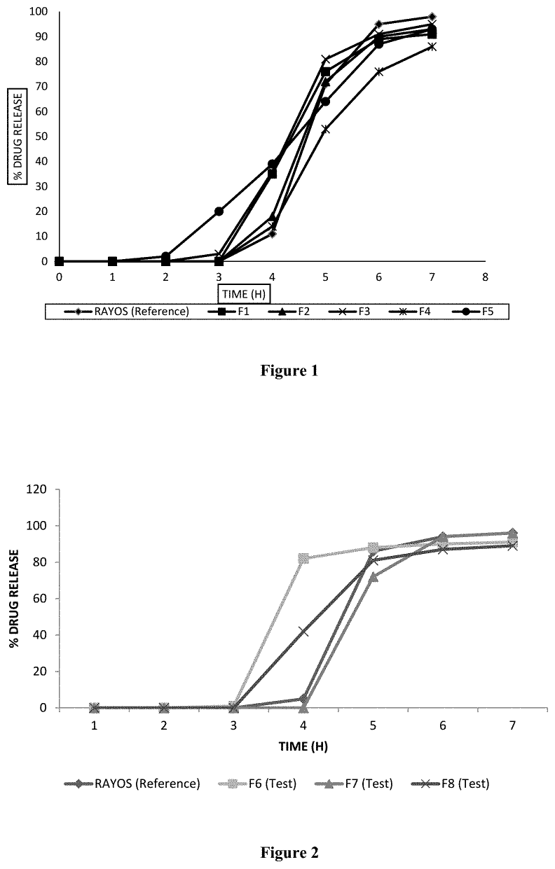

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the definitions herein, may utilize the present invention to its fullest extent. The following specific embodiments are to be construed as merely illustrative, and not limitative of the remainder of the disclosure in any way whatsoever.

Except as defined herein, all the technical and scientific terms used herein have the same meaning as commonly understood by one of ordinary skill in the art to which the invention relates.

### Definitions

For the purpose of the disclosure, listed below are definitions of various terms used to describe the present invention. Unless otherwise indicated, these definitions apply to the terms as they are used throughout the specification and the appended claims, either individually or as part of a larger group. They should not be interpreted in the literal sense. They are not general definitions and are relevant only for this application.

It should be noted that, as used in this specification and the appended claims, the singular forms “a,” “an,” and “the” include plural referents unless the content clearly dictates otherwise.

It should be noted that the term “or” is generally employed in its sense including “and/or” unless the content clearly dictates otherwise.

As used herein, the term “about” means approximately and in the context of numerical values the term “about” can be construed to estimate a value that is ±10% of the value or range recited.

The term “excipient(s)” as used herein means a diluent, binder, disintegrant, glidant, lubricant, coating material or the like, which is non-toxic, and inert, which does not have undesirable effects on a subject to whom it is administered and is suitable for delivering a therapeutically active agent (prednisone) to the target site without affecting the therapeutic activity of the said agent.

As used herein, the term “formulation” or “composition” or “pharmaceutical composition” or “dosage form” as used herein synonymously include solid dosage forms such as granules, multiunit particulate systems (MUPS), pellets, spheres, tablets, capsules, mini-tablets, layered tablets, beads, particles and the like; and liquid dosage forms such as solutions, suspensions, emulsions, colloids and the like, meant for oral administration. The active pharmaceutical compound is prednisone.

Within the context of the present invention and as used herein the term “prednisone” unless indicated otherwise in the entire specification, refers to prednisone in its free form.

Within the context of the present invention and as used herein, unless indicated otherwise, references to total weight of the pharmaceutical composition refers to the total weight of the active agent(s) and pharmaceutically acceptable excipient(s).

Within the context of the present invention and as used herein the term “subject” refers to an animal, preferably a mammal, and most preferably a human. In the context of the present invention, the term “mammal” is used interchangeably with the term “patient” or “subject”. In the context of the present invention, the phrase “a subject in need thereof” means a subject (patient) in need of the treatment of a disease or disorder for which prednisone is used.

Within the context of the present invention and as used herein the term ‘diluent’ refers to an agent used as filler in order to achieve the desired composition volume or weight. The diluent may be present in the pharmaceutical composition in the form of a single compound or in the form of a mixture of compounds. Diluents are often added to tablet formulations to provide better tablet properties such as to improve cohesion, to allow direct compression manufacturing, to enhance flow and to adjust weight of tablet as per die capacity. Diluents are generally classified into three categories namely organic, inorganic and co-processed diluents. The organic diluents include but are not limited to, lactose such as α-lactose monohydrate, spray dried lactose and anhydrous lactose, starch such as potato starch, corn starch or maize starch, and pregelatinized starch, icing sugar with starch, sucrose, mannitol, sorbitol, cellulose such as powdered cellulose and microcrystalline cellulose. The inorganic diluents include but are not limited to calcium phosphates such as anhydrous dibasic calcium phosphate, dibasic calcium phosphate and tribasic calcium phosphate. Some of the insoluble diluents include but are not limited to starch, powdered cellulose, microcrystalline cellulose, calcium phosphate and the like. Some of the soluble diluents include but are not limited to lactose, sucrose, mannitol, sorbitol and the like.

Binders are dry powders or liquid which are added during granulation process to promote granules and cohesiveness. Binders are, but not limited to, cellulose and its derivatives including, ethyl cellulose, hydroxypropyl cellulose, hydroxypropyl methylcellulose (hypromellose), methylcellulose and hydroxyethyl cellulose, carboxymethyl cellulose, gelatin, liquid glucose, corn starch or maize starch, pregelatinized starch, hydrocolloids, sugars, polyvinyl pyrrolidone, sodium alginate, acacia, alginic acid, tragacanth, xanthan, used either alone or combinations thereof.

Disintegrant as used in herein refers to any material that facilitates the break-up of a tablet prepared from the composition when placed in contact with an aqueous medium. Suitable disintegrants include, but are not limited to, crospovidone, sodium starch glycolate, hydroxypropyl starch, microcrystalline cellulose, carboxymethylcellulose sodium or calcium, croscarmellose sodium, pregelatinized starch, polacrilin potassium, low-substituted hydroxypropylcellulose, sodium or calcium alginate, agar, guar gum, chitosan, alginic acid and mixtures thereof.

Glidants improve the flowability of the composition. Exemplary glidants are, but not limited to, fumed silica (colloidal silicon dioxide), colloidal silica, powdered cellulose, talc, tribasic calcium phosphate, magnesium stearate, magnesium carbonate, mixtures thereof and the like.

Lubricants are added in small quantities to tablet formulations to improve certain processing characteristics. The role of the lubricants is to ensure that tablet formation and ejection can occur with low friction between the tablet ingredients and the die walls of the tableting machine. Lubricant prevents sticking to punch faces and enhances product flow by reducing interparticulate friction. The lubricant may be present in the pharmaceutical composition in the form of a single compound or in the form of a mixture of compounds. Lubricants are, but not limited to sodium oleate, sodium stearate, sodium benzoate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc, mixtures thereof and the like.

One or more of these excipients can be selected and used by the artisan having regard to the particular desired properties of the solid dosage form. The amount of each type of excipient employed, e.g. diluent, binder, disintegrant, glidant and lubricant may vary within ranges conventional in the art.

Suitable pharmaceutical compositions include, but are not limited to, capsules, tablets, granules, powders and unit dose pockets. Preferably the oral pharmaceutical composition is a tablet. The tablet can be coated or non-coated.

Coating materials are, but not limited to, sugars, hydroxypropyl methylcellulose (hypromellose), hydroxypropyl cellulose, methylcellulose, ethylcellulose, polyvinyl alcohol, sodium carboxymethylcellulose, calcium pectinate, sodium alginate, alginic acid, povidone, coatings based on methacrylic acid and its esters, such as Eudragit®, mixtures thereof and the like. As alternatives for the above coating materials, sometimes pre-formulated coating products such as those sold as OPADRY™ will be used, for example Opadry White or Opadry Green. The products sold in a solid form require only mixing with a liquid before use. Alternatively, film-forming agents may be applied as powders, using suitable powder coating equipment known in the art.

Seal coating is the first step in the tablet coating process. It provides the initial protection to the core and prevents migration of ingredients from the outer coating layer to the core and vise-versa. Seal coating is performed by the application of a polymer-based coating to the surface of the tablet core. It also helps in improving the stability of the active ingredient in the core by forming a barrier between the core and the functional coating, where functional coating polymer can cause degradation of the active ingredient.

Functional coatings can be used to modify the drug release behavior from the dosage form. Depending on the type of polymers and composition (like pore former, plasticizer etc.) used it is possible to either delay the release of the drug (such as in enteric coatings) or use the coating to sustain the release of the drug from the dosage form over extended periods of time. Functional coating is a complete, sustained release, aqueous coating system utilizing water insoluble polymers like ethylcellulose, polyvinyl acetate as the rate controlling polymer for drug release. The key advantages of using water in soluble polymers for functional coating are:
Rate of drug release is modified by increasing or decreasing the amount of polymer coat applied,
Uniform drug release independent of pH

In an embodiment, the delayed-release pharmaceutical composition comprises of prednisone and one or more pharmaceutical excipient(s).

In another embodiment, the delayed-release pharmaceutical composition comprises of prednisone, one or more diluent(s), one or more binder(s), one or more disintegrant(s), one or more lubricant(s), one or more glidant(s) and one or more coating material(s).

In an embodiment, the delayed-release pharmaceutical composition comprises of prednisone.

In an embodiment, the delayed-release pharmaceutical composition contains prednisone in the range of about 0.1% w/w to about 10% w/w of the composition.

In an embodiment, the pharmaceutically acceptable excipient(s) is selected from the group consisting of diluent, binder, disintegrant, lubricant, glidant, coating material or a combination thereof.

In an embodiment, the diluent is selected from the group consisting of lactose such as α-lactose monohydrate, spray dried lactose and anhydrous lactose, starch such as potato starch, corn starch or maize starch and pregelatinized starch, icing sugar with starch, sucrose, mannitol, sorbitol, cellulose such as powdered cellulose and microcrystalline cellulose, calcium phosphates such as anhydrous dibasic calcium phosphate, dibasic calcium phosphate and tribasic calcium phosphate and the like. The diluent may be used in the range of about 60-90% by weight of the composition.

In an embodiment, the binder is selected from the group consisting of cellulose and its derivatives including, ethyl cellulose, hydroxypropyl cellulose, hydroxypropyl methylcellulose, methylcellulose and hydroxyethyl cellulose, carboxymethyl cellulose; gelatin, liquid glucose; corn starch or maize starch; pregelatinized starch; hydrocolloids; sugars; polyvinyl pyrrolidone, sodium alginate, acacia, alginic acid, tragacanth and xanthan, used either alone or combinations thereof. The binder may be used in the range of about 1-10% by weight of the composition.

In another embodiment, the disintegrant is selected from the group consisting of crospovidone, sodium starch glycolate, hydroxypropyl starch, microcrystalline cellulose, carboxymethylcellulose sodium or calcium, croscarmellose sodium, pregelatinized starch, polacrilin potassium, low-substituted hydroxypropylcellulose, sodium or calcium alginate, agar, guar gum, chitosan, alginic and the like used either alone or in combination thereof. The disintegrant may be used in the range of about 2-15% by weight of the composition.

In another embodiment, the glidant is selected from the group consisting of fumed silica (colloidal silicon dioxide), colloidal silica, powdered cellulose, talc, tribasic calcium phosphate, magnesium stearate, magnesium carbonate and the like used either alone or in combination thereof. The glidant may be used in the range of about 0.5-5% by weight of the composition.

In another embodiment, the lubricant is selected from the group consisting of sodium oleate, sodium stearate, sodium benzoate, sodium chloride, stearic acid, sodium stearyl fumarate, calcium stearate, magnesium stearate, magnesium lauryl sulfate, sodium stearyl fumarate, sucrose esters or fatty acid, zinc, polyethylene glycol, talc, mixtures thereof and the like used either alone or in combinations thereof. The lubricant may be used in the range of about 0.5-3% by weight of the composition.

In an embodiment, the diluent is selected from the group consisting of lactose, starch, microcrystalline cellulose and calcium phosphate or a combination thereof.

In an embodiment, the binder is polyvinyl pyrrolidone.

In an embodiment, the disintegrant is croscarmellose sodium.

In an embodiment, the disintegrant is intragranular and/or extragranular.

In an embodiment, the glidant is fumed silica.

In an embodiment, the glidant, fumed silica is used with either an intragranular or an extragranular disintegrant.

In an embodiment, the lubricant is magnesium stearate.

In an embodiment, the lubricant, magnesium stearate is used with either an intragranular or an extragranular disintegrant.

In an embodiment, the coating material is selected from the group consisting of hydroxypropyl methylcellulose (hypromellose), ethylcellulose, polyvinyl acetate, calcium pectinate, sodium alginate, alginic acid, povidone, Opadry or a combination thereof.

In another embodiment, the delayed-release pharmaceutical composition of the present invention comprises prednisone, icing sugar with starch, lactose monohydrate, microcrystalline cellulose, dicalcium phosphate, croscarmellose sodium, polyvinyl pyrrolidone, fumed silica, magnesium stearate, hydroxypropyl methylcellulose, ethylcellulose and Opadry.

In another embodiment, the composition of the invention can be in standard-release, immediate-release, rapid-onset, delayed-release or dual-release form.

In another embodiment, the composition of the invention is delayed-release form.

### Process for the Preparation of Prednisone Composition:

In an embodiment, the present invention relates to a process for preparing a delayed release pharmaceutical composition containing a therapeutically effective amount of prednisone.

### Process A:

In an embodiment, the process comprises:
1. Dispensing all the raw materials as per the formulation sheet. Co-sifting the intragranular material through suitable sieve;
2. Dissolving binder in purified water to prepare binder solution;
3. Loading the co-sifted material of step 1 in high shear mixer and mixing it for around 15 min;
4. Granulating the dry mix using binder solution of step 2 in high shear mixer and drying the wet granules in fluid bed dryer to achieve target LOD (loss on drying);
5. Sifting and/or milling the dried granules and blending it with extra-granular material in conta blender and lubricating the blend with magnesium stearate;
6. Compressing the lubricated blend using rotary tablet press;
7. Performing the coating on core tablet with appropriate coating parameters using suitable coating material.

### Process B:

In an embodiment, the process comprises:
1. Dispensing all the raw materials as per the formulation sheet. Co-sifting the intragranular material through suitable sieve;
2. Loading the co-sifted material of step 1 in high shear mixer and mixing it for around 45 min;
3. Lubricating the blend with magnesium stearate in high shear mixer and mixing it for around 3 min;
4. Compressing the lubricated blend using rotary tablet press;
5. Performing the coating on core tablets with appropriate coating parameters using suitable coating material.

### Process C:

In an embodiment, the process comprises of steps to be carried out in Part A and Part B as: Part A
1. Dispensing all the raw materials as per the formulation sheet. Co-sifting of intra-granular material as a dry mix through suitable sieve, comprising of prednisone as active ingredient and excipients namely diluent, disintegrant and binder;
2. Dissolving binder in purified water to prepare binder solution;
3. Loading the co-sifted material of step 1 in high shear mixer granulator and mixing for 15 min approximate;
4. Granulating the dry mix using binder solution of step 2 in high shear mixer granulator and drying the wet granules in fluid bed dryer to achieve target LOD;
5. Sifting and/or milling of the dried granules and blending it in conta blender and lubricating the blend referred to as “Active”;

### Part B

6. Co-sifting of intra-granular material as a dry mix through suitable sieve comprising of only the excipients namely diluent, disintegrant and binder, collectively defined as a “Inactive”;
7. Dissolving Hydroxypropyl methylcellulose in purified water to prepare binder solution;
8. Loading the co-sifted material of step 6 in high shear mixer granulator and mixing for 15 min approximate;
9. Granulating the dry mix using binder solution of step 7 in high shear mixer granulator and drying the wet granules in fluid bed dryer to achieve target LOD;
10. Sifting and/or milling of the dried granules of step 9 and blending it in conta blender;
11. Blending of granules of step 5 and granules of step 10 in conta blender for 25 min approximately and lubricating the blend with extra granular material comprising diluent, disintegrant and binder;
12. Compressing the lubricated blend using rotary tablet press with appropriate tooling;
13. Performing coatings on core tablet with appropriate coating parameters using suitable coating material.

In an embodiment, the ratio of the Active to the Inactive ranges from about 1:1 to about 1:100 wherein the composition so formed is stable.

In an embodiment, the ratio of the Active to the Inactive is about 1:1.

In an embodiment, the ratio of the Active to the Inactive is about 1:3.

In an embodiment, the ratio of the Active to the Inactive is about 1:5.

In an embodiment, the ratio of the Active to the Inactive is about 1:10.

In an embodiment, the ratio of the Active to the Inactive is about 1:25.

In an embodiment, the ratio of the Active to the Inactive is about 1:50.

In an embodiment, the ratio of the Active to the Inactive is about 1:100.

In an embodiment, the coating process involves seal coating followed by functional coating.

In an embodiment, the steps for seal coating process comprises of:

(i) Preparing 15% w/w solution of Opadry White or hydroxypropyl methyl cellulose in purified water by dispersing the powder in purified water under stirring.

(ii) The coating solution of step (i) is used to seal coat the tablets.

In an embodiment, the steps for functional coating process comprises of:

(i) Preparing hydroxypropyl methyl cellulose solution by dispersing the powder in purified water under stirring.

(ii) Adding the solution obtained from step (i) in the dispensed aqueous dispersion of Surelease® and further diluting the same to 15% w/w with distilled water.

(iii) The coating solution of step (ii) is used to functional coat the tablets.

In an embodiment, the sifter used for silting the ingredients is vibrator sifter or a sieve.

In another embodiment, the dry blend can be performed in a suitable mixer, such as a container blender, fluid bed dryer, drum blender, v-blender or a high shear mixer.

In an embodiment, the granulating process can be performed using fluidized bed processor, fluid bed top spray granulator or fluidized spray drying.

In an embodiment, tablet compression can be performed in a tablet press.

In an embodiment the coating process can be performed in a coating pan or fluid bed.

In an embodiment, the coating is performed as a seal coating followed by a functional coating.

In an embodiment, the functional coating comprises of water-in soluble polymers and water soluble polymers.

In an embodiment, the ratio of water-insoluble polymer to water soluble polymer is preferably between 55:45 to 95:5, more preferably between 70:30 to 85:15, most preferable 80:20.

In an embodiment, the water in-soluble polymer present in the functional coating plays a vital role for delayed release of the active ingredient.

The compositions of the present invention can be packed into suitable containers such as bottles, blisters or pouch. Further, the packages may optionally contain a desiccant or an antioxidant or oxygen absorbent or combinations thereof.

In an aspect, the present invention relates to use of a delayed-release pharmaceutical composition comprising prednisone as a therapeutic agent for treatment of arthritis, blood disorders, breathing problems, severe allergies, skin diseases, cancer, eye problems, and immune system disorders, wherein the said composition is as described herein above in one or more embodiments of the present invention.

In another embodiment, the present invention relates to a method of treating arthritis, blood disorders, breathing problems, severe allergies, skin diseases, cancer, eye problems, and immune system disorders, comprising administering to a subject in need thereof a therapeutically effective amount of the prednisone composition; wherein the said composition is as described in one or more embodiments of the present invention as described herein above.

In another embodiment, the present invention relates to use of the composition of prednisone, for the manufacture of a medicament for treating arthritis, blood disorders, breathing problems, severe allergies, skin diseases, cancer, eye problems, and immune system disorders; wherein the said composition is as described herein above in one or more embodiments of the present invention.

In another embodiment, the composition of prednisone may be packaged in a suitable container depending upon the formulation and the method of administration of the composition. Suitable containers known to a person skilled in the art include blister pack or bottle pack.

In another embodiment, the present invention provides a pharmaceutical kit comprising prednisone and one or more pharmaceutically acceptable excipient(s). The kit may further comprise a package insert, including information about the indication, usage, doses, direction for administration, contraindications, precautions and warnings.

In another embodiment, the pharmaceutical compositions of the present invention can include all the dosage forms known to a person skilled in art, viz. formulations such as single unit dosage forms in the form of tablets, bilayer tablets, inlaid tablets, tablet in tablet, multilayered tablets, minitablets filled in capsules and the like; beads, pellets presented in a sachet, capsule or tablet capsules such as soft and hard gelatin; lozenges or sachets; granulates, microparticles, multiparticulates, powder and the like.

It is understood that modifications that do not substantially affect the activity of the various embodiments of this invention are included within scope of the invention disclosed herein. Accordingly, the following examples are intended to illustrate but not to limit the scope of the present invention.

### EXAMPLES

TABLE 1
Composition of Prednisone tablets (core tablet comprising
intragranular and extragranular ingredients):

| | Quantity (% w/w) | | |
|---|---|---|---|
| | Batch No. | | |
| Ingredients | F1 | F2 | F5 |

CORE TABLET
Intragranular

| | | | |
|---|---|---|---|
| Prednisone | 0.56 | 0.37 | 0.37 |
| Icing Sugar with starch | 10.00 | 10.00 | — |
| Lactose Monohydrate | — | — | 81.46 |
| Microcrystalline Cellulose | 61.94 | 31.06 | — |
| Dicalcium phosphate | — | 31.06 | — |
| Croscarmellose Sodium | 4.00 | 4.00 | 5.00 |
| Polyvinylpyrrolidone | — | — | 6.67 |
| Fumed silica | — | — | — |
| Magnesium Stearate | — | — | — |

Binder solution

| | | | |
|---|---|---|---|
| Polyvinylpyrrolidone | 7.50 | 7.50 | — |
| Purified Water | q.s. | q.s. | q.s. |

Extragranular

| | | | |
|---|---|---|---|
| Croscarmellose Sodium | 4.00 | 10.00 | 5.00 |
| Microcrystalline Cellulose | 10.00 | 4.00 | — |
| Fumed silica | 1.00 | 1.00 | 1.00 |
| Magnesium Stearate | 1.00 | 1.00 | 0.50 |
| Weight of Core tablet | 100 | 100 | 100 |

SEAL COATING

| | | | |
|---|---|---|---|
| Opadry White 03K580000 | 3.00 | 3.00 | — |
| Opadry White AMB-II 88A180040 | — | — | 3.00 |
| Purified water | q.s. | q.s. | q.s. |
| Weight of Seal coated tablet | 103.00 | 103.00 | 103.00 |

FUNCTIONAL COATING

| | | | |
|---|---|---|---|
| Surelease E-7-19040 | 15 | 10 | 12 |
| HPMC 3 Cps | | | |
| Purified water | q.s. | q.s. | q.s. |
| Weight of functional coated tablet | 118.5 | 113.3 | 115.4 |

### Process for Preparing the Compositions Provided in Table 1:

1. Co-sifting the intragranular material comprising of prednisone, icing sugar with starch, lactose monohydrate, microcrystalline cellulose, dicalcium phosphate, croscarmellose sodium, polyvinylpyrrolidone, fumed silica, and magnesium stearate through suitable sieve;
2. Dissolving binder, polyvinylpyrrolidone in purified water to prepare binder solution;
3. Loading the co-sifted material of step 1 in high shear mixer and mixing it for around 15 min;
4. Granulating the dry mix using binder solution of step 2 in high shear mixer and drying the wet granules in fluid bed dryer to achieve target LOD;
5. Sifting and/or milling the dried granules and blending it with extra-granular material comprising croscarmellose sodium, microcrystalline cellulose, fumed silica, magnesium stearate in conta blender and lubricating the blend with magnesium stearate;
6. Compressing the lubricated blend using rotary tablet press;
7. Performing the coating on core tablet with appropriate coating parameters using suitable coating material and;
8. Packing the coated tablets in bottle pack.

TABLE 2
Composition of Prednisone tablets (core tablet
comprising only intragranular ingredients).

Quantity (% w/w)
Batch No.

| | | |
|---|---|---|
| Ingredients | F3 | F4 |

CORE TABLET
Intragranular

| | | |
|---|---|---|
| Prednisone | 0.37 | 0.37 |
| Lactose Monohydrate | 81.46 | 81.46 |
| Croscarmellose Sodium | 10.00 | 10.00 |
| Polyvinylpyrrolidone | 6.67 | 6.67 |
| Fumed silica | 1.00 | 1.00 |
| Magnesium Stearate | 0.50 | 0.50 |
| Weight of Core tablet | 100.0 | 100.0 |

SEAL COATING

| | | |
|---|---|---|
| Opadry White AMB-II 88A180040 | — | 3.00 |
| HPMC E5 | 4.00 | — |
| Purified water | q.s. | — |
| Weight of Seal coated tablet | 104.00 | 103.00 |

FUNCTIONAL COATING

| | | |
|---|---|---|
| Surelease E-7-19040 | 13 | 12 |
| HPMC 3 Cps | | |
| Purified water | q.s. | q.s. |
| Weight of functional coated tablet | 117.5 | 115.4 |

### Process for Preparing the Compositions Provided in Table 2:

1. Co-sifting the intragranular material comprising of prednisone, icing sugar with starch, lactose monohydrate, microcrystalline cellulose, dicalcium phosphate, croscarmellose sodium, polyvinylpyrrolidone, fumed silica, and magnesium stearate through suitable sieve;
2. Loading the co-sifted material of step 1 in high shear mixer and mixing it for around 45 min;
3. Lubricating the blend with magnesium stearate in high shear mixer and mixing it for around 3 min;
4. Compressing the lubricated blend using rotary tablet press;
5. Performing the coating on core tablets with appropriate coating parameters using suitable coating material and;
6. Packing the coated tablets in bottle pack.

TABLE 3
Dissolution data of different formulations in purified
water in comparison with Reference drug (RAYOS).
Prednisone test tablet is released in Purified Water
environment, under conditions of 500 mL of a
dissolution medium at 37° C. ± 0.5° C., USP
method-II (paddle with sinker), 100 rpm speed wherein
the tablet exhibits a dissolution profile as follows:

| | Formulation | | | | | |
|---|---|---|---|---|---|---|
| Time | RAYOS | F1 | F2 | F3 | F4 | F5 |
| point | (Reference) | (Test) | (Test) | (Test) | (Test) | (Test) |
| (h) | % Drug release | | | | | |
| 1 | 0 | 0 | 0 | 0 | 0 | 0 |
| 2 | 0 | 0 | 0 | 0 | 0 | 2 |
| 3 | 0 | 0 | 0 | 3 | 0 | 20 |
| 4 | 11 | 35 | 18 | 36 | 14 | 39 |
| 5 | 71 | 76 | 72 | 81 | 53 | 64 |
| 6 | 95 | 89 | 90 | 91 | 76 | 87 |
| 7 | 98 | 91 | 93 | 95 | 86 | 93 |

Results: From the above table, it can be found that the test formulations are able to delay the drug release similar to that of reference product.

TABLE 4
Composition of Prednisone tablets (core tablet comprising
intragranular and extragranular ingredients):

Batch No

| | | | |
|---|---|---|---|
| | (F6) | (F7) | (F8) |
| Ingredients | % w/w | % w/w | % w/w |

Part A
Intragranular part A

| | | | |
|---|---|---|---|
| Prednisone | 0.37 | 2.08 | 2.08 |
| Lactose Monohydrate | 9.63 | — | 9.71 |
| Mannitol | — | 9.71 | |
| Microcrystalline Cellulose | 9.62 | 9.71 | 9.71 |
| Croscarmellose Sodium | 1.14 | 3.00 | 3.00 |
| Hydroxypropyl Methyl | 1.14 | — | — |
| Cellulose | | | |
| Binder solution | | | |
| Hydroxypropyl Methyl | 0.40 | 1.00 | 1.00 |
| Cellulose | | | |
| Purified Water | q.s. | q.s. | q.s. |
| Lubrication | | | |
| Stearic Acid | 0.05 | 0.05 | 0.05 |

Part B
Intragranular part B

| | | | |
|---|---|---|---|
| Lactose Monohydrate | 33.75 | — | 32.63 |
| Mannitol | — | 32.63 | |
| Microcrystalline Cellulose | 33.75 | 32.62 | 32.62 |
| Croscarmellose Sodium | 4.00 | — | 3.00 |
| Binder solution | | | |
| Hydroxypropyl Methyl | 5.40 | 2.00 | 2.00 |
| Cellulose | | | |

Extra granular

| | | | |
|---|---|---|---|
| Croscarmellose Sodium | — | 6.00 | 3.00 |
| Stearic Acid | 0.75 | 1.20 | 1.20 |
| Weight of core tablet | 100.00 | 100.00 | 100.00 |

Seal Coating

| | | | |
|---|---|---|---|
| Hydroxypropyl Methyl | 4.00 | 4.00 | 4.00 |
| Cellulose | | | |
| Weight of seal coated | 104.00 | 104.00 | 104.00 |
| tablet | | | |

Functional Coating

| | | | |
|---|---|---|---|
| Ethyl cellulose aqueous | 10.40 | 7.64 | 13.31 |
| dispersion | | | |
| Hydroxypropyl Methyl | 2.60 | 1.45 | 3.33 |
| Cellulose | | | |
| Polyethylene glycol 6000 | — | 0.36 | |
| Weight of functional coated | 117.00 | 113.45 | 120.64 |
| tablets | | | |

### Process for Preparing the Compositions Provided in Table 4:

### Part A

1. Co-sifting of intra-granular material comprising of Part A dry mix through suitable sieve;
2. Dissolving Hydroxypropyl methylcellulose in purified water to prepare binder solution;
3. Loading the co-sifted material of step 1 in high shear mixer granulator and mixing for 15 min approximate;
4. Granulating the dry mix using binder solution of step 2 in high shear mixer granulator and drying the wet granules in fluid bed dryer to achieve target LOD;
5. Sifting and/or milling of the dried granules and blending it in conta blender and lubricating the blend with stearic acid;

### Part B

6. Co-sifting of intra-granular material comprising of Part B dry mix through suitable sieve;
7. Dissolving Hydroxypropyl methylcellulose in purified water to prepare binder solution;
8. Loading the co-sifted material of step 6 in high shear mixer granulator and mixing for 15 min approximate;
9. Granulating the dry mix using binder solution of step 7 in high shear mixer granulator and drying the wet granules in fluid bed dryer to achieve target LOD;
10. Sifting and/or milling of the dried granules of step 9 and blending it in conta blender;
11. Blending of granules of step 5 and granules of step 10 in conta blender for 25 min approximately and lubricating the blend with extra granular material;
12. Compressing the lubricated blend using rotary tablet press with appropriate tooling;
13. Performing coatings on core tablet with appropriate coating parameters using coating material as per formulation table 1.
14. Packing the coated tablets in bottle pack.

TABLE 5
Dissolution data of different formulations in purified
water in comparison with Reference drug (RAYOS).
Prednisone test tablet is released in Purified Water
environment, under conditions of 500 mL of a
dissolution medium at 37° C. ± 0.5° C., USP
method-II (paddle with sinker), 100 rpm speed wherein
the tablet exhibits a dissolution profile as follows:

Formulation

| | | | |
|---|---|---|---|
| RAYOS | F6 | F7 | F8 |
| (Reference) | (Test) | (Test) | (Test) |

| | |
|---|---|
| Time (h) | % Drug release |

| | | | | |
|---|---|---|---|---|
| 1 | 0 | 0 | 0 | 0 |
| 2 | 0 | 0 | 0 | 0 |
| 3 | 0 | 1 | 0 | 0 |
| 4 | 5 | 82 | 0 | 42 |
| 5 | 86 | 88 | 72 | 81 |
| 6 | 94 | 90 | 94 | 87 |
| 7 | 96 | 91 | 96 | 89 |

Results: From the above table, it can be found that the test formulations are able to delay the drug release similar to that of reference product.

TABLE 6
Composition of Prednisone tablets (core tablet
comprising only intragranular ingredients):

| | Batch No. |
|---|---|
| | F9 |
| Ingredients | % w/w |
| CORE TABLET | |
| Prednisone | 1.00 |
| Lactose Monohydrate | 77.50 |
| Croscarmellose Sodium | 15.00 |
| Polyvinylpyrrolidone | 5.00 |
| Colloidal silicon dioxide | 1.00 |
| Magnesium Stearate | 0.50 |
| Weight of Core tablet | 100.00 |
| SEAL COATING | |
| HPMC E5 | 4.00 |
| Purified water | q.s. |
| Weight of Seal coated tablet | 104.00 |
| FUNCTIONAL COATING | |
| Surelease E-7-19040 | 12.00 |
| HPMC 3 Cps | 3.00 |
| Purified water | q.s. |
| Weight of coated tablet | 119.00 |

Process for preparing the compositions provided in Table 6:
1. Co-sifting the intragranular material comprising of Lactose monohydrate, Croscarmellose sodium, Polyvinylpyrrolidone and colloidal silicon dioxide through suitable sieve;
2. Loading the co-sifted material of step 1 in high shear mixer and mixing it for around 5 min;
3. Mixing the API (Prednisone) with step 2 blend in high shear mixer for around 40 min;
4. Lubricating the blend with magnesium stearate in high shear mixer and mixing it for around 3 min;
5. Compressing the lubricated blend using rotary tablet press;
6. Performing the coating on core tablets with appropriate coating parameters using suitable coating material and;
7. Packing the coated tablets in bottle pack.

TABLE 7
Stability data of Prednisone tablets at initial
and 1 week open petri plate (50° C./75% RH)

| Batch No. | F1 | F6 | F9 |
|---|---|---|---|
| Initial | 0.64 | 0.26 | 0.52 |
| 1 Week_50° C./75% RH | 6.18 | 0.41 | 2.01 |
| (open petri plate) | | | |

Conclusion: Formulation F6 and F9 were found to be more stable than formulation F1 after 1 week (50° C./75% RH) open petri plate stability study.

We claim:

1. A delayed-release pharmaceutical composition comprising prednisone, one or more diluent(s), one or more binder(s), one or more disintegrant(s), one or more lubricant(s), one or more glidant(s) and one or more coating material(s).

2. A process for the preparation of a delayed-release pharmaceutical composition comprising prednisone, one or more diluent(s), one or more binder(s), one or more disintegrant(s), one or more lubricant(s), one or more glidant(s) and one or more coating material(s).

3. The process as claimed in claim 2, comprising of steps being carried out in Part A and Part B as:
Part A
1. Dispensing all the raw materials as per the formulation sheet. Co-sifting of intra-granular material as a dry mix through suitable sieve, comprising of prednisone as an active ingredient and excipients namely diluent, disintegrant and binder;
2. Dissolving binder in purified water to prepare binder solution;
3. Loading the co-sifted material of step 1 in high shear mixer granulator and mixing for 15 min approximate;
4. Granulating the dry mix using binder solution of step 2 in high shear mixer granulator and drying the wet granules in fluid bed dryer to achieve target LOD;
5. Sifting and/or milling of the dried granules and blending it in conta blender and lubricating the blend referred to as “Active”;
Part B
6. Co-sifting of intra-granular material as a dry mix through suitable sieve comprising of only the excipients namely diluent, disintegrant and binder, collectively defined as a “Inactive”;
7. Dissolving binder in purified water to prepare binder solution;
8. Loading the co-sifted material of step 6 in high shear mixer granulator and mixing for 15 min approximate;
9. Granulating the dry mix using binder solution of step 7 in high shear mixer granulator and drying the wet granules in fluid bed dryer to achieve target LOD;
10. Sifting and/or milling of the dried granules of step 9 and blending it in conta blender;
11. Blending of granules of step 5 and granules of step 10 in conta blender for 25 min approximately and lubricating the blend with extra granular material comprising diluent, disintegrant and binder;
12. Compressing the lubricated blend using rotary tablet press with appropriate tooling;
13. Performing coatings on core tablet with appropriate coating parameters using suitable coating material.

4. The process as claimed in claim 3, wherein the ratio of the Active to the Inactive ranges from about 1:1 to about 1:100 and the composition so formed is stable.

5. The process as claimed in claim 2, wherein the composition is formulated into a tablet using a coating technique which has significant impact on drug release.

6. The process as claimed in claim 2, wherein the coating is performed as a seal coating followed by a functional coating.

7. The process as claimed in claim 2, wherein the functional coating comprises of water-in soluble polymers and water soluble polymers.

8. The process as claimed in claim 2, wherein the ratio of water-insoluble polymer to water soluble polymer is preferably between 55:45 to 95:5, more preferably between 70:30 to 85:15, most preferable 80:20.

9. The process as claimed in claim 2, wherein the delayed release of the active ingredient depends on the water in-soluble polymer present in the functional coating.

10. A method for treating or preventing various conditions, diseases, disorders, comprising administering to a subject in need thereof composition of claim 1 in an amount effective to treat or prevent a condition, a disease or a disorder.
